(19) United States
(12) Patent Application Publication
(10) Pub. No.: US 2019/0013093 A1
(43) Pub. Date: Jan. 10, 2019

(54) SYSTEMS AND METHODS FOR ANALYZING HEALTHCARE DATA

(71) Applicant: Arizona Board of Regents on Behalf of the University of Arizona, Tucson, AZ (US)

(72) Inventors: Marvin J. Slepian, Tucson, AZ (US); Fuad Rahman, Santa Clara, CA (US); Arijit Mitra, Santa Clara, CA (US)

(21) Appl. No.: 15/746,767

(22) PCT Filed: Jul. 20, 2016
(86) PCT No.: PCT/US16/43175

§ 371 Date: Jan. 22, 2018

Related U.S. Application Data
(60) Provisional application No. 62/194,920, filed on Jul. 21, 2015.

(51) Int. Cl. G16H 40/20; G16H 10/20; G06F 17/21; G06F 17/27; G06N 5/04; G16H 10/60

(52) U.S. Cl. CPC G16H 40/20; G16H 10/20; G16H 10/60; G06F 17/2785; G06N 5/046; G06F 17/21

(57) ABSTRACT

Systems, methods, and software products analyze healthcare data. First input data is collected from a first source and second input data is collected from a second source disparate from the first source. The second source has a data format that is different from a format of the first source. The first input data is processed to determine a first concept and the second input data is processed to determine a second concept. A relationship between the first and second concepts is determined. The first and second concepts are stored within a knowledgebase based upon the relationship and a patient medical model is generated from the knowledgebase.

### RELATED APPLICATION

This application claim priority to U.S. Patent Application Ser. No. 62/194,920, titled “Systems and Methods for Analyzing Healthcare Data”, filed Jul. 21, 2015, and incorporated herein in its entirety by reference.

### BACKGROUND

In modem healthcare computerization, the doctor is often restricted as to what information may be provided to, and is available within, healthcare computers and other digital information systems. Healthcare computers and the modern range of digital devices mostly provide data entry forms that require manual information entry in a certain format and within a certain space; for example, the doctor uses a keyboard to type or dictate entries into a predefined textual data field. The amount of time the doctor is allotted for each patient is driven by many issues which have changed over the years, including: increasing patient load, the rise in chronic disease conditions and economic circumstances, such as to enable insurance payments for each patient. Thus, the doctor typically has an increasing burden of patient number coupled with less time to spend on each patient and the amount of available data entry into the electronic medical records is reduced. In the past, physicians would spend 30-60 minutes on a typical office encounter. This is now reduced to 10 minutes in the U.S. on average, and even less in several other countries around the world. Similarly rounding in the hospital or clinic, or even in the home of field as a house call, is typically shorter today than in years past.

The role of the health care encounter—whether it be the office, clinic, hospital, home or field, or any other location in which care is delivered, is critical in obtaining relevant information to steward, guide and otherwise direct the delivery of care and enhance the accuracy of care. Studies have repeatedly demonstrated over the years that despite the increased availability of complex, sophisticated diagnostic devices, instruments, lab tests, imaging systems and the like, that it is the history taking, the physician or health worker asking of questions—as to symptoms and signs, that is the most significant element in moving care forward. Studies have clearly demonstrated that more than 70% of diagnoses and advancement of care steps emanate from physician or health worker questioning of the patient. As such, about seventy percent of proper diagnoses for the patient are made by the doctor using non-computerized information, such as: what the patient says, how the patient looks and acts, how the patient behaves, how the patient sits, how the patient walks, how the patient smells, and other information gained by the doctor during one-on-one patient encounters and consultations. But this information is not known by the healthcare computers or other digital or other data systems. For example, where the same doctor consults with the patient on consecutive occasions, it is the doctor's memory and mental vision and reconstruction of previous consultations that helps the most in determining whether the patient's health is deteriorating, changing, or improving, and whether current treatment is effective. Where different doctors consult with the patient, information from previous consultations is often not available and the newly on board physician has a less complete picture of the patient.

Modern healthcare is made available today through many unconnected services that collectively provide care to a patient. Each service collects and stores its data for future use, but only some of which is shared with other services. Further, as discussed herein, and in the accompanying filing “Patent 1” there is much valuable data—e.g. patient appearance, sound and smell that is presented, available, and valuable with a patient encounter though presently largely perceived by the health care provider, and is not transduced for capture and digital transformation. Much information that each service collects is also unusable by other services as the data is often from different contexts, and is in a format not easily transferred and assimilated. Because data is essentially “siloed”, key factors in caring for the patient are often lost, resulting in additional procedures, additional hospital visits, and additional costs for both the patient and healthcare organizations.

### SUMMARY

In one embodiment, a method analyzes healthcare data. First input data is collected from a first source and second input data is collected from a second source disparate from the first source. The second source has a data format that is different from a format of the first source. The first input data is processed to determine a first concept and the second input data is processed to determine a second concept. A relationship between the first and second concepts is determined. The first and second concepts are stored within a knowledgebase based upon the relationship and a patient medical model is generated from the knowledgebase.

In another embodiment, a method analyzes healthcare data. Input data is received from a plurality of disparate sources. Text is extracted from the input data and processed using natural language processing (NLP) to determine a plurality of concepts, each concept being based upon understanding and sentiment derived from the text. A relationship between each of the concepts is determined and high level concepts are derived from the plurality of concepts. Each of the concepts and the high level concepts are stored within a database based upon the relationship. The input data is processed to determine concepts relating to healthcare. The information in each of the concepts is normalized and direct concepts are extracted from the healthcare data by using NLP, semantic analysis, and inference extraction. Derived concepts are derived from the direct concepts and the direct concepts and the derived concepts are stored within in a concept bank to form a knowledgebase.

In another embodiment, a system analyzes healthcare data. The system includes a plurality of transducers operable to collect healthcare data from disparate sources, a natural language processing (NLP) and semantic engine for identifying direct concepts in the healthcare data, a converter, implemented as machine readable instruction executed by a digital processor, for receiving and converting the healthcare data to form a database of information associated with the patient, and an analyzer, implemented as machine readable instruction executed by a digital processor, for processing the database to generate a health status of the patient.

In another embodiment, a software product has instructions, stored on non-transitory computer-readable media, wherein the instructions, when executed by a computer, perform steps for analyzing healthcare data. The instructions include instructions for collecting first input data from a first source, instructions for collecting second input data from a second source disparate from the first source, the second source having a data format that is different from a format of the first source, instructions for processing the first input data and the second input data to determine a first concept and a second concept, respectively, instructions for determining a relationship between the first and second concepts, instructions for storing the first and second concepts within a knowledgebase based upon the relationship, and instructions for generating a patient medical model from the knowledgebase.

In another embodiment, a software product has instructions, stored on non-transitory computer-readable media, wherein the instructions, when executed by a computer, perform steps for analyzing healthcare data. The instructions include instructions for receiving input data from a plurality of disparate sources, instructions for extracting text from the input data, instructions for processing the text using natural language processing (NLP) to determine a plurality of concepts, each concept based upon understanding and sentiment derived from the text, instructions for determining a relationship between each of the concepts, instructions for storing each of the concepts and the high level concepts within a database based upon the category, instructions for deriving high level concepts from the plurality of concepts, instructions for storing each of the concepts and the high level concepts within a database based upon the category, instructions for processing the input data to determine concepts relating to healthcare, instructions for normalizing the information in each of the concepts, instructions for extracting direct concepts from the healthcare data by using NLP, semantic analysis, and inference extraction, instructions for deriving derived concepts from the direct concepts, and instructions for storing the direct concepts and the derived concepts in a concept bank to form a knowledgebase.

### BRIEF DESCRIPTION OF THE FIGURES

FIG. 1 shows one exemplary system for analyzing healthcare data, in an embodiment.

FIG. 2 shows the system of FIG. 1 in further exemplary detail.

FIG. 3 shows exemplary construction of the concept of FIG. 2 from the input data of FIG. 1, in an embodiment.

FIG. 4 shows the analyzer of FIG. 1 with a predictor that interacts with an information portal interface to receive a query from an interrogator, in an embodiment.

FIG. 5 is a schematic showing exemplary generation of the concept graph of FIG. 4 by the predictor.

FIG. 6 is a schematic illustrating exemplary initialization of a phrase extraction and concept recognition tool, in an embodiment.

FIG. 7 is a schematic illustrating exemplary core semantic algorithms for generating concept, phrase, metadata, relationships, and patient data, in an embodiment.

FIG. 8 shows exemplary categorization of words and concepts.

FIG. 9 shows exemplary operation of the NLP and semantic engine of FIG. 2, in an embodiment.

FIG. 10 is a schematic illustrating exemplary automatic update of the knowledgebase of FIG. 2 by an event processing engine, in an embodiment.

FIG. 11 is a flowchart illustrating one exemplary method for analyzing healthcare data, in an embodiment.

FIG. 12 is a schematic illustrating exemplary automatic update of the knowledgebase of FIG. 2 by an event processing engine, in an embodiment.

FIG. 13 shows the knowledgebase of FIG. 12 with exemplary data illustrating the ability to add concepts on the fly, and the need to create events to add concepts within a new category, in an embodiment.

FIG. 14 is a flowchart illustrating one exemplary method for initializing the system of FIG. 1, in an embodiment.

FIG. 15 is a flowchart illustrating one exemplary method for updating the knowledgebase of FIG. 2, in an embodiment.

FIG. 16 shown one exemplary framework for implementing the healthcare analytic engine of FIGS. 1 and 2 using an Apache Spark platform, in an embodiment.

### DETAILED DESCRIPTION OF THE EMBODIMENTS

A concept aggregation model and other extensions are added to a large-scale analytical platform to analyze medical information to derive meaningful relationships within the information from the disparate data sources. Captured data, resulting from many different contexts, is transformed and stored as concepts that are normalized values of the captured data that may be used within models, comparisons, and so on.

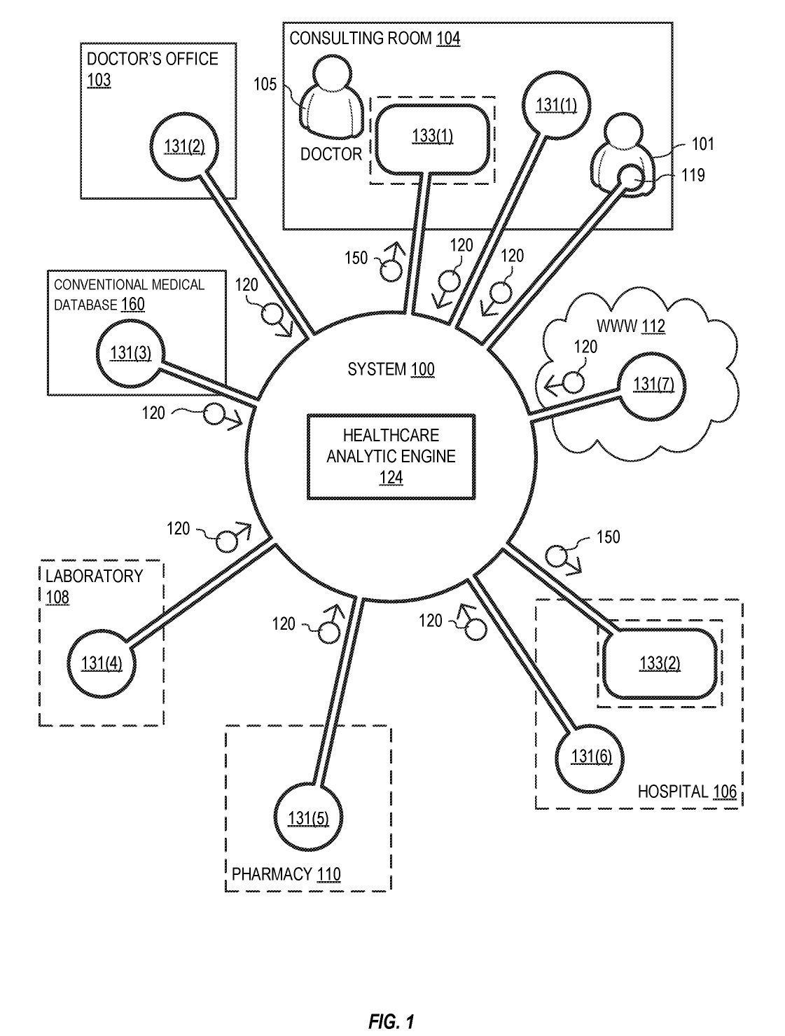

FIG. 1 shows one exemplary system 100 for analyzing healthcare data. System 100 is for example a distributed computer that receives input data 120 from a plurality of transducers 131 that operate to collect healthcare information from disparate sources, such as a consulting room 104 of a doctor 105, a hospital 106, a laboratory 108, a pharmacy 110, a conventional electronic medical record database 160, the World Wide Web (WWW) 112. For example, input data 120 may include one or more of audio data (e.g., sensed sounds from within consulting room 104), image data (e.g., video and/or still images captured by a camera within consultation room 104), textual data (e.g., data entered by doctor 105 and/or patient 101), and measurement data (e.g., measurements from a medical device coupled with, implanted into, and/or worn by patient 101). In embodiments, data 120 includes information relevant to a medical condition which is presently not being captured into the medical record to enhance documentation, aid diagnosis, provide population big data information and guide therapy. This data may be sensory, mobility and/or dynamic data—e.g. a clear odor, a tremulous movement, an audible respiratory noise, a visual grimace, an affect—and may include asked data, evoked data, detected data, symptom data, sign data, lab data, imaging data, test data, as well as sensory data. This data maybe described and termed as “symptom and sign Metadata”—in the sense that this data may relate to a given symptom or sign—for example a patient may complain of reduced exercise capacity and upon walking in the office has a noticeably reduced gait, stride length and speed of walking—none of this typically enters the medical record.

System 100 operates to collect and store input data 120 for a plurality of patients (i.e., not just patient 101). That is, system 100 operates to collect “big-data”.

Transducers 131 include one or more input devices (e.g., sensors such as one or more of: a microphone, a camera, a scanner, an olfactory sensor, a taste sensor, a touch sensor, a temperature sensor, a stiffness/roughness sensor, and so on) for collecting healthcare data in many different formats. Transducer 131 may be mobile such that (a) a doctor may take transducer 131 on house calls to collects input data 120 during consultations at remote locations, and/or (b) the patient may take transducer 131 home such that input data 120 is collected in a home environment of patient 101, and/or (c) transducer 131 may be implemented within other apparatus (e.g., a mobile phone, a fitness tracker, etc.) and is transported and/or worn by the patient.

In one example of operation, transducer 131(1) collects audio data from within consulting room 104. In another example of operation, transducer 131(2) uses an imaging device to collect medical images within hospital 106. In another example of operation, transducer 131(7) includes a plurality of web scrappers to collect healthcare information from WWW 112. Transducers 131 may be mobile and configured within medical monitoring equipment (e.g., a blood pressure monitor worn by the patient, a sensor implanted into the patient) such that input data 120 is collected from patient 101 at any location.

In another example of operation, transducer 131(3) uses a procedure configured with a conventional medical record database 160 to capture electronic medical records (EMRs). In another example of operation, transducer 131(4) includes a data capture port for collecting medical information (e.g., test details and test results) from within a laboratory 108. In another example of operation, transducer 131(5) collects medical information (e.g., filled prescriptions, drug purchases) within a pharmacy. In another example of operation, transducer 131(7) collects medical information from social media. For example, transducer 131(7) may generate medical information 224 from posts and tweets made by patient 101. Similarly, where patient 101 wears a tracking type device 119 that collects movement and other medical related information of patient 101, transducer 131(7) interacts with a corresponding social media account on WWW 112 and collects input data 120. Device 119 may also represent a portable medical device that periodically measures blood pressure of patient 101 within a defined period, wherein one or more transducers 131 may wirelessly connect with device 119 to collect the measured data. In one embodiment, patient 101 may have one or more implanted sensors that provide input data 120 to system 100. For example, transducer 131 may include one or more implanted or wearable sensors that provide input data 120 to system 100.

System 100 includes a healthcare analytic engine 124 that processes information input data 120 to generate one or more patient medical models 133. In the example of FIG. 1, system 100 generates patient medical model 133(1) for display within consulting room 104 during the consultation between doctor 105 and patient 101. Patient medical model 133 provides an enhanced view of the health of patient 101 as derived from input data 120 collected and stored within system 100. Patent medical model 133 may also be displayed and stored within the patient's EHR.

Healthcare analytic engine 124 is a big data analytical engine that generates patient medical model 133 defining a current health status of patient 101 based upon one or more of patient sentiment, patient mood, patient general wellbeing, patient morale, patient activity, and social graph, inferred from input data 120. Further, healthcare analytic engine 124 generates patient medical model 133 to include predicted events for patient 101 based upon past events and current health status of patient 101 and stored outcomes and events for other patients having similar past events and health status to patient 101.

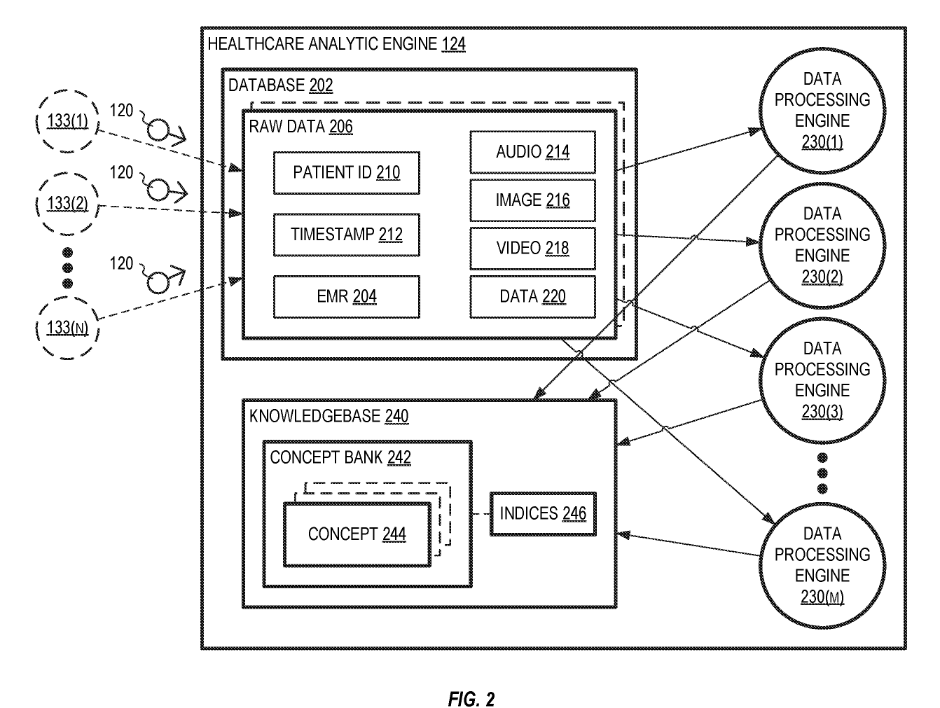

FIG. 2 shows healthcare analytic engine 124 of system 100, FIG. 1, processing input data 120 to generate a knowledgebase 240. As noted above, transducers 131 operate to collect input data 120 for a plurality of patients and from disparate data sources. System 100 processes input data 120 from disparate sources and thereby collects healthcare information that was typically lost by prior art systems and methods. System 100 then transforms input data 120 from its raw format (e.g., audio from a microphone, images of notes, video of the patient meeting with the doctor, etc.) into a format that is usable by system 100. As noted above, input data 120 may be of any of a plurality of digital formats, including digitized audio, images, scanned notes, measurements, and so on. As input data is received by healthcare analytic engine 124 it is stored within a database 202 as raw data 206 for example. It should be noted that traditional EMRs cannot and do not collect and store this type of audio and image data. Database 202 may be implemented as one of Oracle and xBD. Further, database 202 may be external to, but accessible by, healthcare analytic engine 124.

Raw data 206 may include a patient ID 210 and a timestamp 212, for example, as received within input data 120. Patient ID 210 identifies one patient (e.g., patient 101) that is associated with the input data 120 and timestamp 212 defines a time that input data 120 was captured. Depending on the format of the data within input data 120, raw data 206 may include one or more of EMRs 204, audio data 214, image data 216, video data 218, and other data 220. For example, audio data 214 may include captured audio from consulting room 104, image data 216 may include images of notes made by doctor 105, video data 218 may include video captured within consulting room 104, and other data 220 may include measured values of blood pressure, weight, and so on.

In one example of operation, transducer 131(7) is implemented as one or more web scrappers that collect healthcare information from www 112. Input data 120(7), received from transducer 131(7), is often unstructured (typically in HTML format) and is stored as other data 220 within raw data 206. In another example of operation, transducer 131(3) is implemented as a database procedure that collects healthcare information from conventional medical record database 160. Input data 120(3), received from transducer 131(3), may already be in the form of EMRs and thereby structured, but may still not be directly evaluated against other EMRs 204.

As shown in FIG. 2, Healthcare analytic engine 124 includes a plurality of data processing engines 230(1-N) that process raw data 206 to generate normalized concepts 244 that are stored within a concept bank 242 to form a knowledgebase 240. Knowledgebase 240 thereby contains concepts 244 determined from a plurality of disparate sources, where each data source provides raw data from completely different contexts that have different dimensions, units and forms. As such, numbers, values, and readings within raw data 206 are typically incompatible with one another. Data processing engines 230(1-N) operate to normalize these numbers, values, and readings into a single normalized and scaled matrix to form concepts 244. Normalization is required to combine and compare “features” collected from multiple disparate sources. To allow these features to contribute to the final computational model, values are normalized against a range and scale defined by the matrix. Each concept 244 defines at least one piece of normalized information determined from raw data 206.

Knowledgebase 240 also includes one or more indices 246 that facilitate rapid searching and processing of stored concepts 244. Knowledgebase 240 is, for example, an XML database such as xDB from EMC, or in the case of big-data processing may be an Hbase database within the Hadoop framework. Knowledgebase 240 concurrently stores structured and non-structured data, handles SQL and NoSQL types of queries, and in one embodiment is implemented as both a MongoDB for unstructured data and an xDB for XML data. In another embodiment, knowledgebase 240 is implemented as an InfoFrame Elastic relational Store (IERS) from NEC that provides a compromise between storing structured and unstructured data.

Each data processing engine 230 is specifically configured for processing certain types of raw data 206. For example, data processing engine 230(1) may be configured to extract concepts 244 from audio data 214 using speech recognition and natural language processing. Data processing engine 230(2) may be configured to extract concepts 244 from image data 216 to using optical character recognition and natural language processing. Data processing engine 230(3) may be configured to extract concepts 244 from video data 218 using one or more of facial recognition, gait recognition, posture recognition, and so on. Knowledgebase 240 thereby contains concepts 244 of normalized data that may be aggregated and collectively evaluated.

In one example of operation, speech by patient 101 within consulting room 104 is captured as audio data 214. Data processing engine 230(1) first converts audio data 214 into text using speech recognition (see speech recognizer 410 of FIG. 1). Then, data processing engine 230(1) processes the text using a natural language processing (NLP) (see NLP and semantic engine 404) to determine language such that the meaning of the speech is understood. Data processing engine 230(1) creates concepts 244 that contain the normalized meaning of the speech. Further, data processing engine 230(1) analyzes audio data 214 to determine tone and cadence of the speech and thereby derives concepts 244 for sentiment and/or mood of patient 101. Data processing engine 230(1) thereby normalizes audio data 214 into concepts 244 that may be compared and understood by system 100, thereby allowing system 100 to understand speech.

In another example of operation, video data 218 is captured of patient 101 within consulting room 104. Data processing engine 230(3) analyzes video data 218 to identify the face of patient 101, and then further analyzes features and metrics of the identified face to determine facial expressions that show the mood of patient 101. For example, over five hundred metrics may be determined from a human face. Texture features may also be recorded, such as skin color, skin texture, eye color, as so on. Data processing engine 230(3) compares these metrics with metrics of known faces (i.e., previously recognized, tagged, or learned faces with known expressions and moods) to determine the expression and mood of patient 101. Data processing engine 230(3) thereby normalizes video data 218 and stores concepts 244 of the identified facial expressions and determined moods. These mood concepts may be (a) be compared to other mood concepts, such as identified in audio data 214, and (b) used to provide a better understanding of patient 101 by system 100. Data processing engine 230(3) may also determine other concepts 244 from identified faces. For example, metrics determined from known sets of faces of a particular age range (e.g., 10-15 years, 15-25 years, and so on) may be used by data processing engine 230(3) to determine age of patient 101 by correlating facial metrics of patient 101 with the grouped metrics of the known faces for each age range. For example, where patient 101 looks older that indicated in their medical records, healthcare analytic engine 124 may indicate that anomaly. Thus, system 100 may operate similarly to a doctor, who first looks at the patient to determine a general wellbeing of the patient as a first impression. Similarly, metrics determined from known sets of faces for male and female people may be used by data processing engine 230(3) to determine gender of patient 101 based upon correlation.

Audio data 214 and video data 218 may contain other contextual information that may be discerned by healthcare analytic engine 124. Where patient 101 is accompanied by another person (e.g., spouse, child, mother, etc.) to consulting room 104, by collecting that information (stored as concepts 244), healthcare analytic engine 124 may provide context to other concepts 244 determined within consulting room 104. For example, if patient 101 is elderly and accompanied by their daughter, the information they provide to doctor 105, or even measurements (e.g., BP) made by the doctor, may be different than when patient 101 visits doctor 105 without the daughter. Similarly, “white coat syndrome” may also be identified, where BP measured by doctor 105 is different than BP measured by a nurse. Thus, by knowing the context of the concepts collected within consisting room 104, healthcare analytic engine 124 may identify, understand, ignore, and/or correct for discrepancies. Where this context information is not present, these discrepancies may be interpreted as changes in the patient's health. Context of patient 101 when staying in hospital 106 may also affect measurements and perceived demeanor of patient 101. Thus, knowing the context of captured information provides a higher quality and consistency of data within the captured information.

As described in Appendix A of U.S. Patent Application Ser. No. 62/194,920, raw medical data is captured that would otherwise be lost. However, unless this data is transformed into a useful format, it is of little use by other applications as described in Appendix B of U.S. Patent Application Ser. No. 62/194,920, least of all because of the volume of such data. Data processing engines 230 operate to transform raw data 206 into derivatives, stored as concepts 244, which are of a format (i.e., data type) that may be compared and used within patient modeling. Advantageously, information within the raw data may overlap, thereby reinforcing the information extracted from the raw data. In the example above, mood concepts are derived from both audio data 214 and video data 218. Based upon the timestamp 212 of raw data 206 containing the data, these mood concepts may reinforce understanding of the mood of patient 101 while within consulting room 104. Although a doctor may observe a patient's expression during a consultation, the expression is typically not recorded, other than in the memory of the doctor, and is therefore typically lost once the doctor and patient separate.

Since concepts 244 are derived from disparate sources and result in similar understanding of patient 101, system 100, and in particular healthcare analytic engine 124, becomes more robust since correlation of the same information derived from multiple sources improves understanding of patient 101.

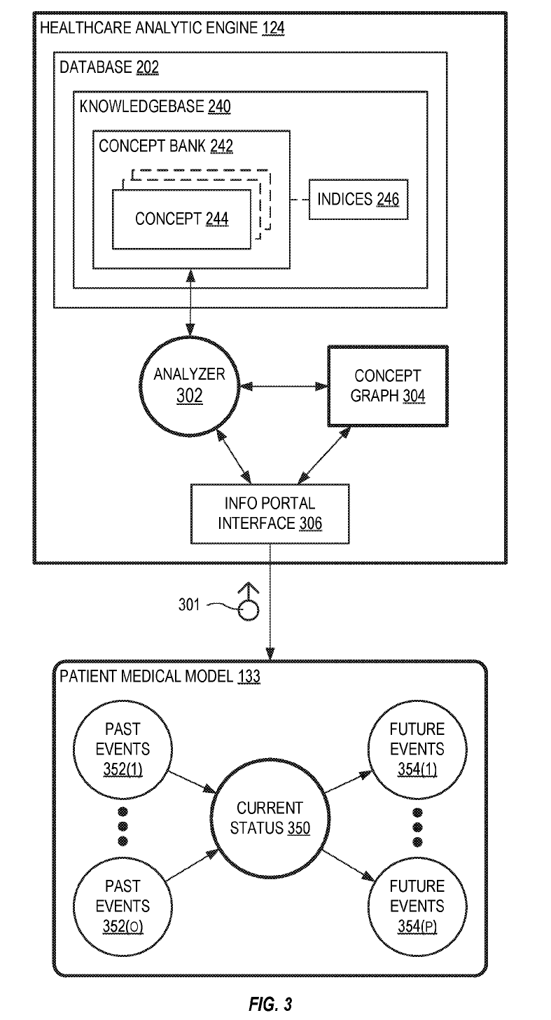

FIG. 3 is a schematic illustrating exemplary operation of healthcare analytic engine 124 to generate patient medical model 133. Healthcare analytic engine 124 includes an analyzer 302 that processes knowledgebase 240 to generate a concept graph 304 from concepts 244 based upon a query 301 received via an information portal interface 306. Query 301 is received from doctor 105 for example when patient 101 enters consulting room 104 and contains an ID of patient 101. Concept graph 304 contains certain concepts 244 of concept bank 242 that are relevant to patient 101 and derived concepts, as described in detail below.

Information portal interface 306 then generates patent medical model 133 for patient 101 based from concept graph 304, where model 133 defines a current medical status 350 of patient 101, zero, one or more past medical events 352(1)-(O) of patient 101, and one or more possible future medical events 354(1)-(P) of patient 101. Patient medical model 133 thereby provides a very powerful and useful medical model of patient 101 that may be used to predict future events based upon actions of patient 101 and actions and results of other patients having similar medical status and taking similar actions. For example, future events 354(1) is a prediction based upon patient 101 conforming to a prescribed medical treatment (e.g., takes a prescribed medication), and future event 354(2) (not shown) is based upon patient 101 not conforming to the prescribed medical treatment. Another future event 354 may be based upon patient 101 having a medical treatment (e.g., a surgery), and so on. Since these predicted future events 354 are also based upon actual outcomes, stored within knowledgebase 240, of other patients having similar medical conditions and taking, or not taking, similar actions, patient medical model 133 provides doctor 105 with a very powerful and accurate prediction of what could happen to patient 101.

Where data from disparate sources results in concurring concepts 244, patient medical model 133, generated from correlation of concepts 244, is improved in quality, accuracy, and confidence.

Although patient medical model 133 is primarily used to predict the effect, or lack of, treatment on patient 101, patient medical model 133 may also be used to predict the effectiveness and/or expected life or durability of an intervention. For example, where patient 101 suffers from peripheral artery disease, a physician may recommend insertion of a stent into the artery. However, the durability or “life” of the stent—i.e., its freedom from thrombosis, migration, fracture and restenosis, is dependent upon many factors, such as gait, amount of movement, nature of movement—e.g., bending, frequency of motion, blood flow, temperature, edema, compliance with anti-thrombotic medication, weight, infection, and so on. To effectively predict the therapeutic lifespan of the stent, all these parameters would need to be measured continually for the patient, which is, of course, impractical. However, healthcare analytic engine 124 may correlate many characteristics of patient 101 with other patients having similar conditions. For example, where patient 101 is diabetic, from the Indian subcontinent, aged about 60, then by correlating other patients having similar characteristics, and medical conditions, healthcare analytic engine 124 may select information from these matched patients to predict the lifespan of the stent within patient 101 based upon the actual life of the same or similar stents in these other patients. Thus, patient medical model 133 may be used to select a more suitable treatment based upon statistical data of past use. Patient medical model 133 may also be used to assess risk to the patient for a particular treatment.

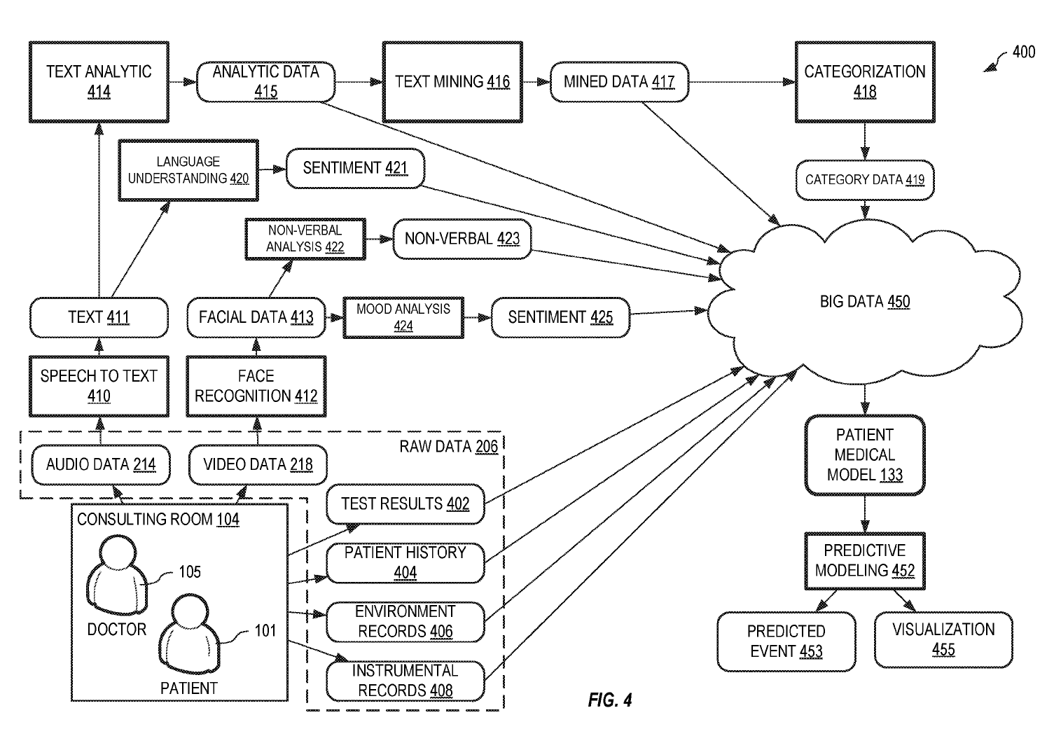

FIG. 4 is a conceptual diagram 400 illustrating exemplary processing of raw data 206 to create big data 450. Big data 450 represents storage and analytical processing of large quantities of data. Raw data 206 includes audio data 214 and video data 218, as described above with reference to FIG. 2, and may further include test results 402, patient history 404 (e.g., in the form of EMRs 204 retrieved from external databases), environment records 406 (e.g., environmental conditions that relate to patient 101), and instrumental records 408 (e.g., measurements such as weight, height, BP, and so on, taken from patient 101), and may include other types of data without departing from the scope hereof. In particular, FIG. 4 shows exemplary processing to extract and normalize concepts from audio data 214 and video data 218. Other steps, not shown for clarity of illustration, process test results 402, patient history 404, environmental records 406, and instrumental records 408.

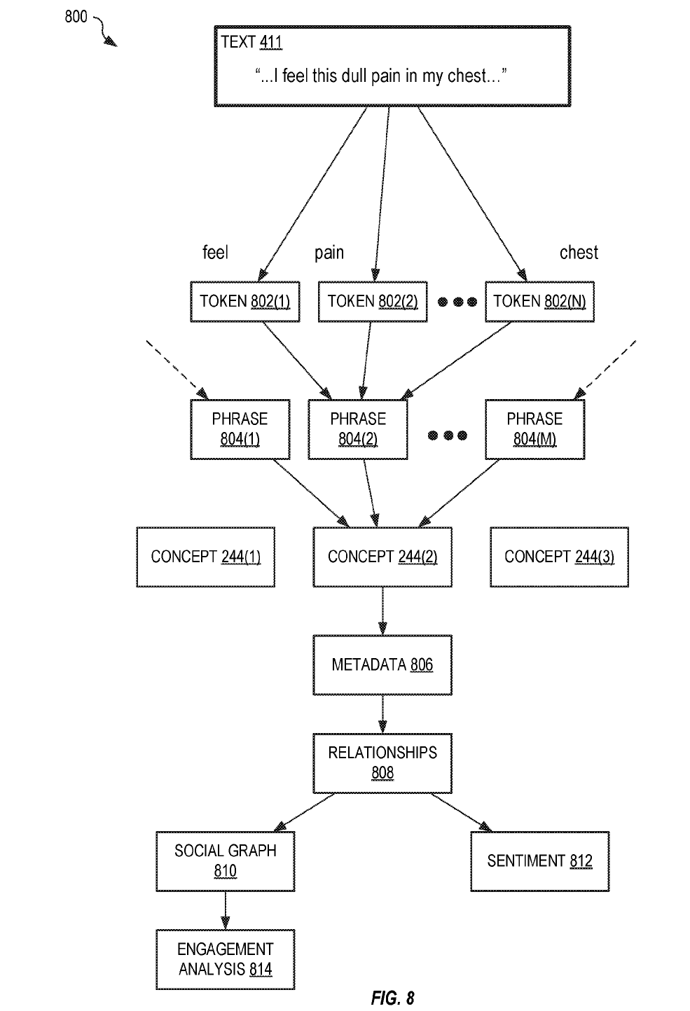

A speech to text process 410 converts audio data 214 into text 411. A text analytic process 414 then converts text 411 into analytic data 415 that includes concepts 244. For example, text analytic process 414 may parse text 411 to form tokens (e.g., tokens 802, FIG. 8) that may combine to form phrases (e.g., phrases 804, FIG. 8). A text mining process 416 processes analytic data 415 to generates mined data 417. For example, mined data 417 may include metadata (e.g., metadata 806, FIG. 8) that defines meaning of concepts found by text analytic process 414. A categorization process 418 identifies categories within the mined data 417 to generate category data 419 that defines relationships (e.g., relationships 808, FIG. 8). Categorization process 418 functions at a higher level than NLP and may be considered as operating more in the area of extraction. Text 411 is also processed by a language understanding process 420 to determine sentiment data 421.

A face recognition process 412 processes video data 218 to generate facial data 413 that may include metrics of identified faces within video data 218. A non-verbal analysis process 422 processes the facial data 413 to generate non-verbal data 423 that may include gait metrics, cadence, hydration, sweating, nutritional status, and so on. Facial data 413 is also processed by a mood analysis process 424 that determines sentiment data 425 from facial expression, including, anxiety state, depression, sadness, fear, confusion and happiness.

Big data 450 is then used to generate patient medical model 133, which may be used by a predictive modeling process 452 to generate a predicted event 453 and/or visualization 455. For example, predicted event 453 is determined for patient 101 based upon one or more of (a) a current medical status of patient 101, a medical history of patient 101 (e.g., as determined from patient history 404), and results/outcomes of patients with similar medical history, current medical status, environmental conditions, and so on, stored within big data 450. For example, based upon results for other similar patients, big data 450 may be used to predict events (i.e., predicted event data 453) that may happen to patient 101 depending on whether certain interventions are followed, or not followed, for patient 101.

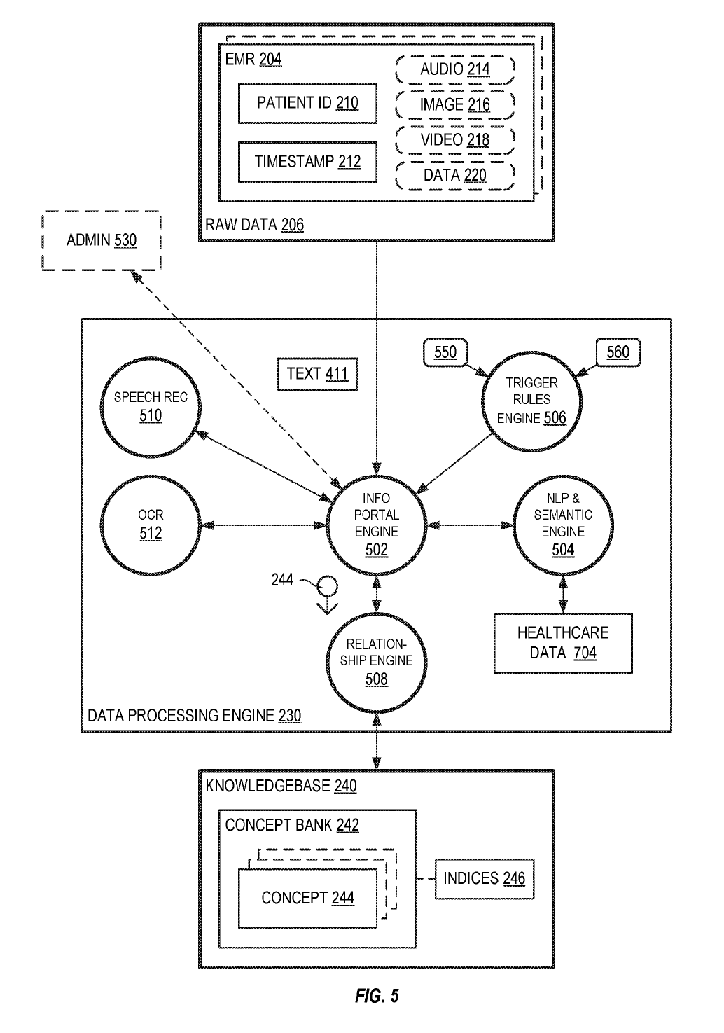

FIG. 5 shows operation of one data processing engine 230 of FIG. 2 in further exemplary detail. In the example of FIG. 5, data processing engine 230 processes natural language found in raw data 206 to generate one or more concepts 244. Data processing engine 230 includes an information portal engine 502 that receives raw data 206 and invokes one or more of a speech recognizer 510, an optical character recognizer 512, and other tools known in the art to convert audio data 214, image data 216, and other data 220 into textual form, illustratively shown as text 411. Information portal engine 502 may then use a trigger rules engine 406 and a natural language processing (NLP) and semantic engine 504 for identifying concepts 244 based upon generic language structure, natural language and semantics found within text 411. Trigger rules engine 506 uses language rules 550 that define structure for the language in which text 411 is defined and operate to facilitate parsing of text. Trigger rules engine 506 may also use a healthcare taxonomy 560 that defines words and phrases of text 411 that are of interest within the field of healthcare. Data processing engine 230 also includes relationship engine 508 that stores concept 244 within concept bank 242 relative to other concepts 244 already therein.

In one embodiment, information portal engine 502 also interacts with an admin terminal 530 to resolve any non-convertible phrases into concepts, wherein system 100 thereby learns and stores new semantics for future processing.

By generating concepts 244, data processing engine 230 normalizes raw data 206 such that concepts 244 may be successfully evaluated and compared against one another. In one example of semantic analysis, where raw data 206 contains video data 218 showing patient 101 smiling and behaving in a relaxed manner, data processing engine 230 may accordingly generate a first concept 244 indicating that patient 101 is happy. Similarly, where raw data 206 contains image data 216 of notes made by doctor 105 that state that patient 101 appears to be jovial, data processing engine 230 may accordingly generate a second concept 244 indicating that patient 101 is happy. Thus, first concept 244, determined from video data 218, and second concept 244, determined from image data 216, (a) both reinforce the determination that patient 101 is happy, and (b) are in a format where they can be easily evaluated and compared. That is, the information has been normalized and stored as concepts that allow evaluation and comparison. As with the above example, by collecting and normalizing data from disparate sources, the normalized information allows system 100 to generate patient medical model 133 that is stronger that using only prior art medical records.

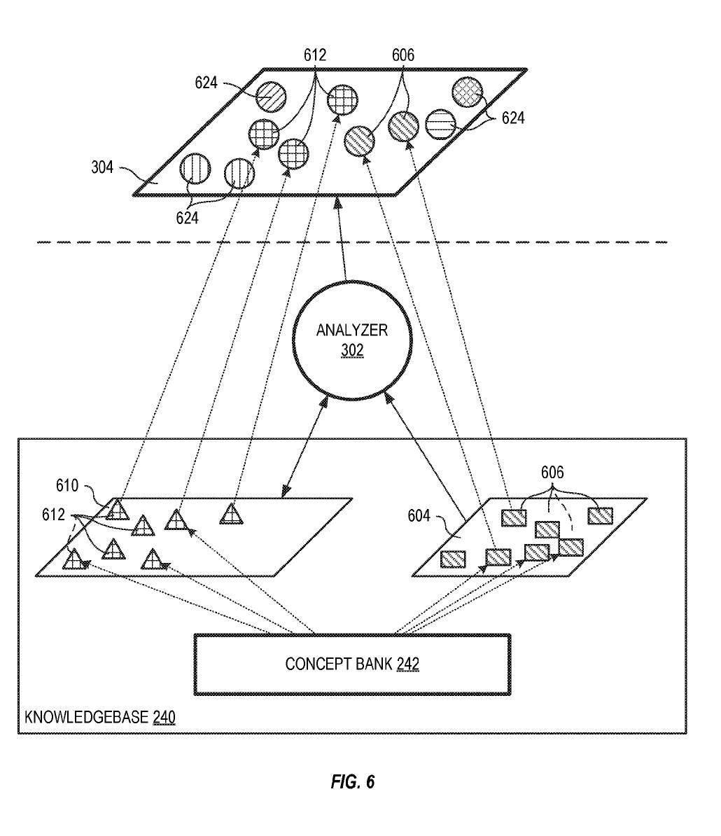

FIG. 6 is a schematic showing exemplary generation of concept graph 304 of FIG. 3 by analyzer 302. Knowledgebase 240 contains a plurality of concepts 244, some of which are direct concepts 606, illustratively shown on a graph 604, that are determined directly from raw data 206, FIG. 2. Knowledgebase 240 also contains a plurality of derived concepts 612, illustratively shown on a graph 610, that are derived by analyzer 302 and/or information portal engine 502 and NLP and/or semantic engine 504 from non-conventional data collected by transducers 131 and/or direct concepts 606. For example, healthcare concepts 612 may represent healthcare information determined from notes of doctor 105 and audio captured within consulting room 104. Healthcare concepts 612 contain one or more of sentiment, location, context, and timing/behavior demographic information. Analyzer 302 then selects certain concepts 244, 606, 612 for use in concept graph 304. For example, analyzer 302 may select all concepts relating to patient 101 and other patients having similar symptoms, treatments, and conditions for example. Further, analyzer 302 operates to derive (e.g., using big data analytical techniques) additional concepts 624 from concepts 244 within knowledgebase 240 for use in concept graph 304. Concept graph 304 thereby provides more complete information that is relevant to patient 101 than available in prior art systems.

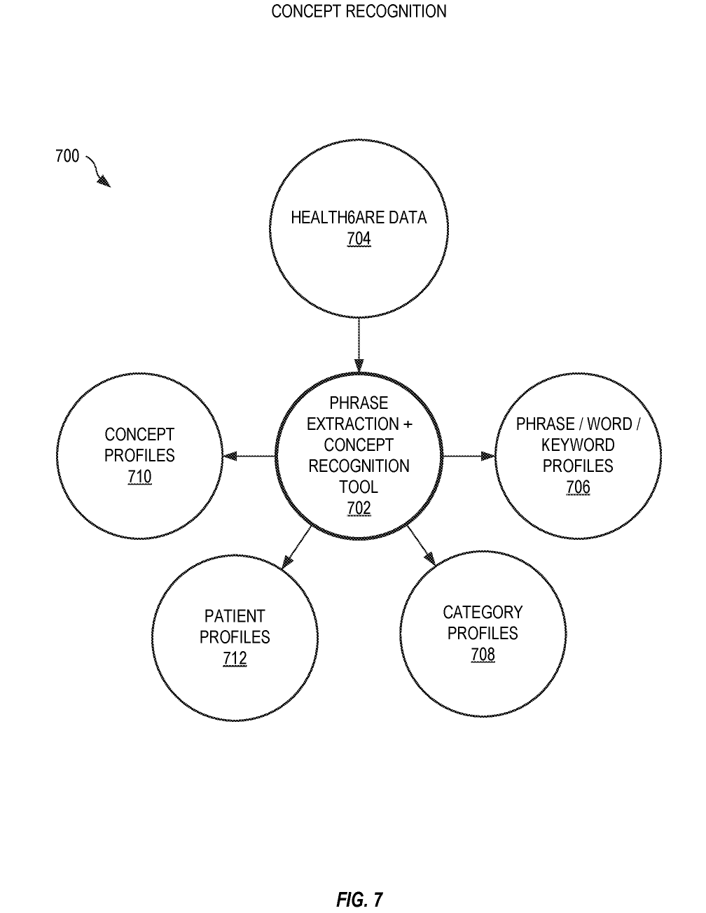

FIG. 7 is a schematic 700 illustrating exemplary initialization of a phrase extraction and concept recognition tool 702. Phrase extraction and concept recognition tool 702 is implemented within NLP and semantic engine 504 and operates to determine concepts 244 by understanding natural language related to healthcare found within input data 120. However, to be able to identify concepts 244 specific to healthcare, phrase extraction and concept recognition tool 702 first processes healthcare data 704 to “learn” phrase/word/keyword profiles 706, category profiles 708, concept profiles 710, and patient profiles 712, that specifically relate to the healthcare area.

Healthcare data 704 is a source of generic healthcare information and may represent one or more of the following: thesaurus/ontologies, Unified Metadata Language Systems (UMLS), Freebase, a concept relationship store, Freebase content, DMOZ links, and Interactive Advertising Bureau (IAB) categories.

An ontology is a declarative model of a domain that defines and represents the concepts existing in that domain, their attributes, and the relationships between them. (See for example, www.openclinical.org/ontologies.html) A number of these clinical ontologies are available in open source. Healthcare analytic engine 124 is configured to incorporate and/or utilizes these ontologies directly or indirectly. For example, where an ontology is available as a database, this database is downloaded and incorporated within healthcare analytic engine 124 for use by phrase extraction and concept recognition tool 702.

UMLS is a well-known research area in medical language understanding sponsored by US National Library of Medicine (NLM) which is a part of National Institute of Health (NIH). See for example www.nlm.nih.gov/research/umls/about_umls.html.

Freebase is a community created database of well-known people, places, and other “things”. For example, there are currently over forty-six million topics. See for example www.frebase.com. Data from Freebase may be downloaded into healthcare analytic engine 124, and/or Freebase may be queried “on-demand” by healthcare analytic engine 124. Although Freebase contains over two billion facts, only a faction are relevant to the medical domain. However, it is an invaluable tool for understanding human speech, the references used, the current or historical analogues used when trying to express their ideas. Freebase is therefore an essential tool for NLP accuracy and understanding.

DMOZ (see www.dmoz.org) derived from the Open Directory Project (ODP) and is the largest, most comprehensive, human-edited directory of the WWW. DMOZ organizes information available on the WWW into a set of predefined categories that healthcare analytic engine 124 uses with NLP to aid in understanding the content of natural conversation between patient 101 and doctor 105.

IAB (see www.iab.net) is a membership body of about six hundred and fifty leading technology and media companies that sell, distribute and optimize digital advertising and marketing. Together, these companies represent approximately eighty-six percent of active advertising in the United States of America. NLP is used extensively in targeting online advertising. IAB has researched, and continues to research, ways to categorize web sites and web content so that the most relevant advertisement is selected to target the current audience. Thus, IAB is a very valuable resource in understanding natural language and is a great tool to better characterize a patient-doctor conversation in its natural form.

Once phrase extraction and concept recognition tool 702 has processed healthcare data 704, phrase/word/keyword profiles 706, category profiles 708, concept profiles 710, and patient profiles 712 are used by NLP and semantic engine 504 to analyze text determined from raw data 206.

Phrase/word/keyword profiles 706 provide healthcare meaning to each word, or group of words (phrase). For example, phrase/word/keyword profiles 706 allow NLP and semantic engine 504 to determine special meaning of two words (tokens) that appear together within a sentence. That special meaning may be different from the meaning of each word individually. For example, in two sentences, provided by patient 101 for example, that use the words “blood” and “clot” together: (a) “I am worried about my blood, am I at risk to bleed and can't clot” as compared to (b) “I am concerned about a blood clot.” In the first sentence (a) the issue is bleeding and inability to clot, whereas in the second sentence (b), the issue is heightened risk for clotting. In another example, each of two statements use the words “heart” and “attack” together: (c) “I was attacked on the street and feel stress. Will this hurt my heart? I should really exercise right now, right??” as compared to (d) “I have a history of heart attack and need to exercise.” In the first sentence (c) the patient is worried about their heart because of an attack, whereas in the second sentence (d) the patient is worried about a heart attack. Category profiles 708 may be used to provide context to, and determine relationships between, concepts 244 and thereby increase understanding of these concepts. Concept profiles 710 are used to build and maintain a list of possible concepts (e.g., based upon healthcare descriptors) that are used to analyze existing records and to compare new records with existing records. Patient profiles 712 are built and maintained to define a descriptive extraction of a patient that uniquely identifies the patient in terms of discriminating features. Collectively, profiles 706, 708, 710, and 712, may be categorized so that groups of patients who have demonstrated similar features may be considered as a “cluster” for statistical and other processing.

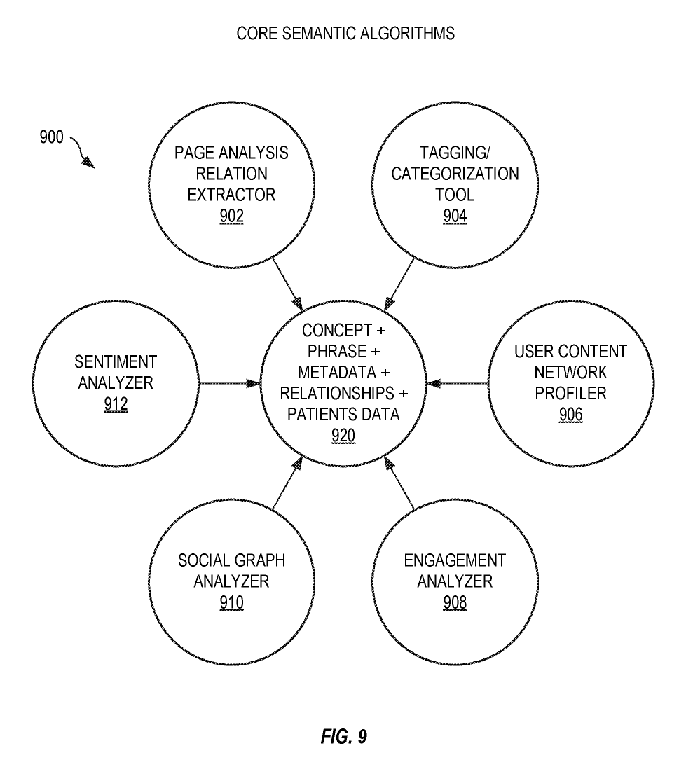

FIG. 8 shows exemplary processing of text 411 of FIG. 5 by NLP and semantic engine 504 to determine concept 244. FIG. 9 is a schematic illustrating exemplary core semantic algorithms 900 for generating concept, phrase, metadata, relationships, and patient data 920. Core semantic algorithms 900 are implemented within one or more data processing engines 230 of FIG. 2, for example. FIGS. 8 and 9 are best viewed together with the following description.

NLP and semantic engine 504 first parses text 411 to generate tokens 802, and then, based upon phrase/word/keyword profiles 706, groups these tokens 802 into possible phrases 804. These factors 804 are then grouped to form concept 244. For example, text 411 may represent captured audio from consulting room 104, wherein patient 101 complains of chest pain. For example, text 411 may include: “I have noticed a decrease in my exercise capacity. I am short of breath with just walking a few blocks. I used to be able to walk six blocks without resting but now I can only walk one block before I need to rest. I don't have a fever, cough, or sputum production. When I walk I also get chest pressure, but not pain.” This example contains the tokens: exercise and capacity. For this contextual concept, these words are used together, since these tokens have meaning for heart failure and indicate a key symptom. Taken alone, these words would be out of context. As the patient explains that he is short of breath (SOB), NLP and semantic engine 504 may add collected characteristics on speech cadence, facial expression, respiratory rate, opening of mouth, mouth breathing, wheezing, huffing/puffing, facial color—pink vs ashen, pallid, etc.

Describing the decline in walking of “Blocks” may be used to provide a gauge of decline intensity and may be standardized. Also these words also provide context indicating that this is in fact a heart failure description, which is further reinforced by patient stating he does not have fever, cough and sputum production—which would steer the context towards pneumonia and infection. Similarly, since the patient states that he does not have chest pain, leads away from acute coronary disease. However, by stating that he does have chest pressure, indicates that we should consider whether there is worsening of coronary artery disease.

Core semantic algorithms 900 include a page analysis relation extractor 902, a tagging/categorization tool 904, a user content network profiler 906, an engagement analyzer 908, a social graph analyzer 910, and a sentiment analyzer 912. One or more of page analysis relation extractor 902, tagging/categorization tool 904, and user content network profiler 906, is invoked within NLP and semantic engine 504 to determine metadata 806 from text 411. Engagement analyzer 908 is then invoked within NLP and semantic engine 504 to determine relationships 808. NLP and semantic engine 504 then invokes social graph analyzer 910 to generate a social graph 810 from concepts 244, metadata 806, and relationships 808. Social graph 810 defines relationships between patients that show similar features (see for example patient profiles 712). Sentiment analyzer 912 is invoked by NLP and semantic engine 504 to generate sentiment 812. Social graph 810 may then be processed to generate engagement analysis 814 that defines how patients are engaged with their doctors. For example, engagement analysis 814 is based upon one or more of body language, gestures, sentiment and mood of the patient when engaging with the doctor. Engagement analysis 814 provides a key indication of successful treatment, giving credence to the age old idea that if the patient likes the doctor, the treatment works better. Concepts 244, phrases 804, metadata 806, and relationships 808 are then used to form concept, phrase, metadata, relationship and patient data 920.

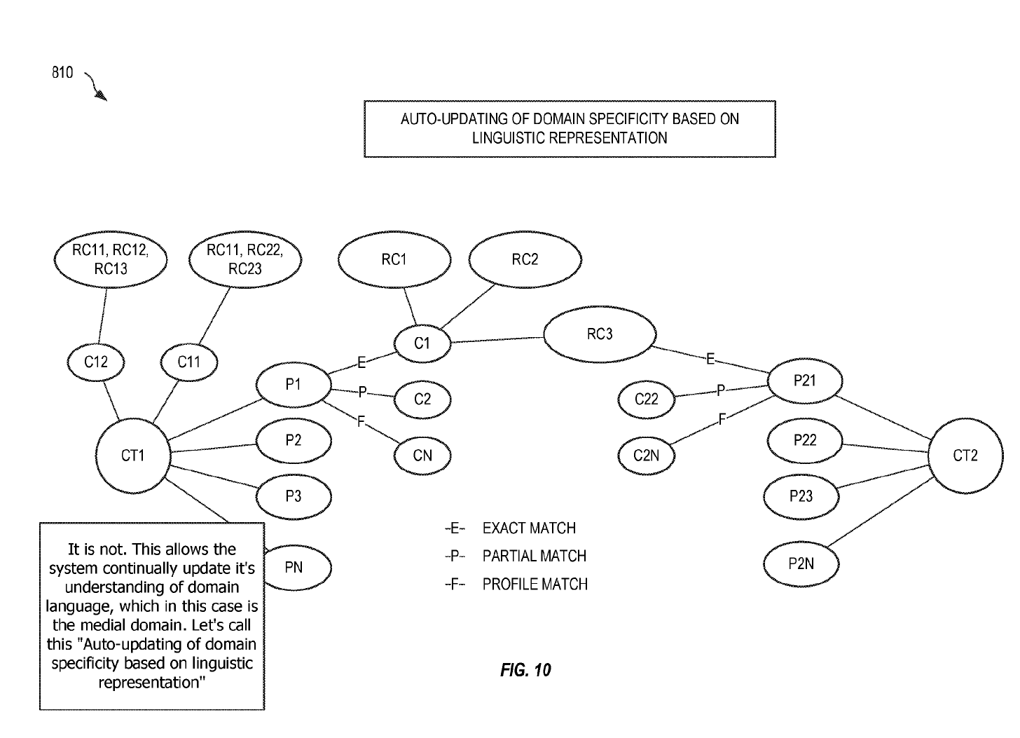

FIG. 10 shows exemplary operation of NLP and semantic engine 504 of FIG. 5 to categorize phrases 804 and concepts 244 to form and maintain social graph 810 of FIG. 8. In the example of FIG. 10, Cn and Cnn represent concepts 244, Pn and Pnn represent phrases 804, CTn represent categories 708, and RCnn represent related concepts 244. Joining lines marked “E” indicate exact matches, joining lines marked “P” indicate partial matches, and joining lines marked “F” indicate profile level matches where a phrase matches a specific profile but does not exactly or partially match a phrase.

NLP and semantic engine 504 uses social graph 810 to determine relationships between these phrases 804 and concepts 244 within a specific category. These relationships are often quoted in normalized weight factors. Social graph 810 allows healthcare analytic engine 124 to continually update its understanding of language used within the medial domain, and may be considered as “auto-updating of domain specificity based on linguistic representation.”

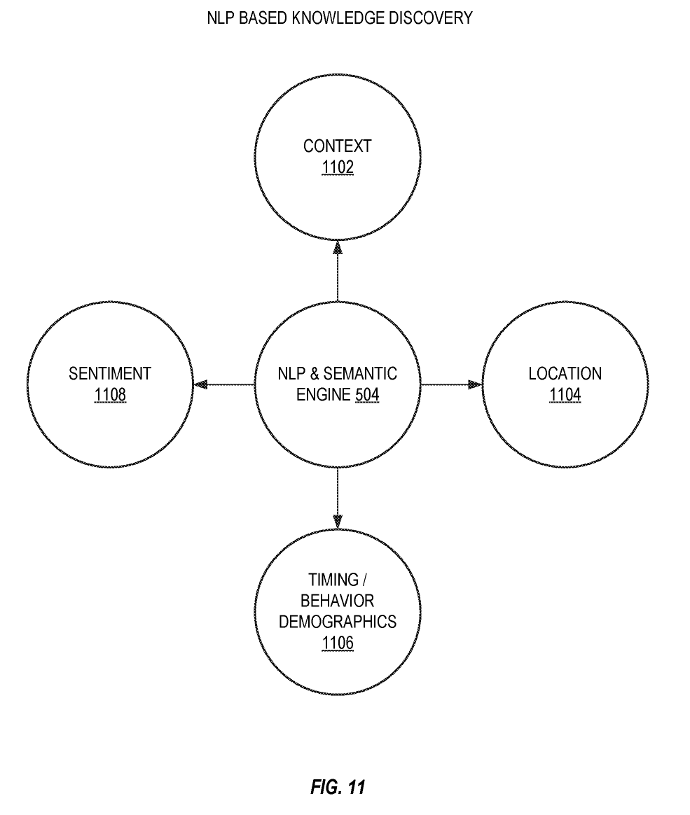

FIG. 11 shows exemplary operation of NLP and semantic engine 504 of FIG. 5. NLP and semantic engine 504 uses one or more algorithms to implement one or more of: phrase extraction, concept recognition, concept connectivity, relation extraction, tagging/categorization, sentiment analysis, and social graph analysis. NLP and semantic engine 504 processes input data 120 to generate concepts 244 that have associated context 1102, associated location 1104, associated timing/behavior demographics 1106, and associate sentiment 1108. By providing context 1102, location 1104, timing/behavior demographics 1106 and associate sentiment 1108 for each concept 244. NLP and semantic engine 504 allows system 100 to derive more information from healthcare data found within input data 120 than is available in prior art methods and systems.

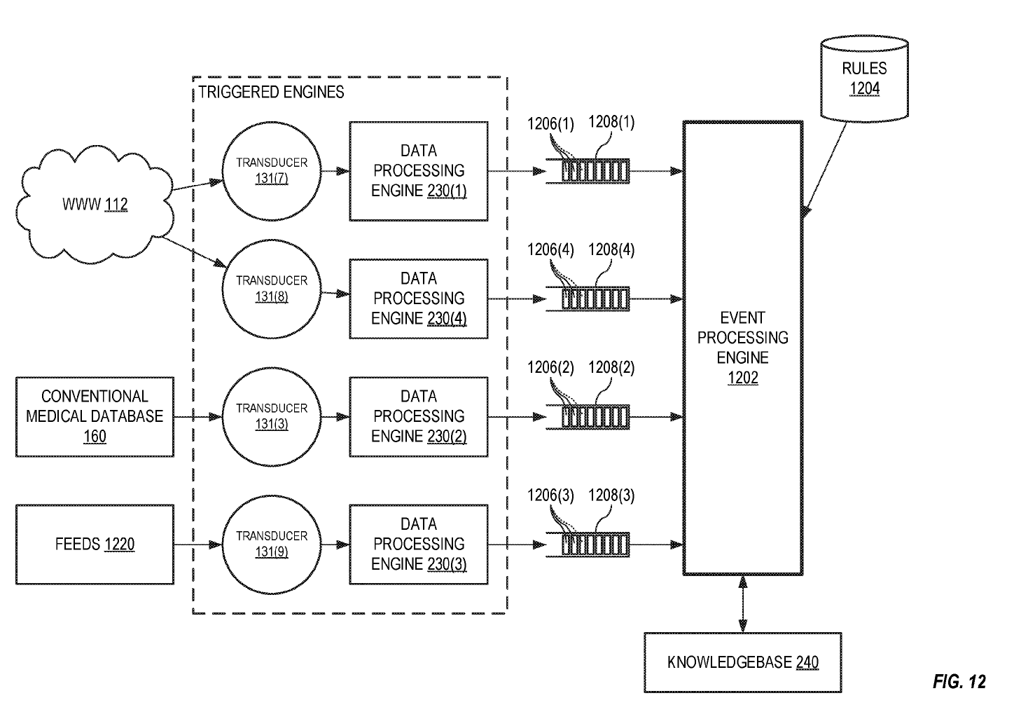

FIG. 12 is a schematic illustrating exemplary automatic update of knowledgebase 240 of system 100, FIG. 2, by an event processing engine 1202. Transducers 131 operate to collect input data 120 (i.e., additional healthcare information) from the disparate healthcare data sources substantially continuously. As shown, transducer 131(7) collects information from www 112. Transducer 131(3) collects information from conventional medical record database 160. In one embodiment, transducer 131(3) is at least in part configured with conventional medical database 160 to retrieve new and/or updated medical information as it is written to conventional medical record database 160. Transducer 131(9) is configured to collect healthcare information from one or more feeds 1220 that represent one or more of live data feeds, RSS feeds, and so on. Additional transducers 131(8), as compared to transducers 131 used during initialization of system 100, may be implemented to collect healthcare information from additional sources, such as within www 112 for example. Each transducer 131 sends input data 120 to a corresponding data processing engine 230 for further analysis.

Each data processing engine 230 process input data 120 and attempts to store the concepts 244 within knowledgebase 240. However, where concepts 244 determined from input data 120 cannot be stored within an existing category or context of knowledgebase 240, data processing engine 230 generates and adds an event 1106 to a corresponding event queue 1108. That is, new concepts 244 may be added to existing categories and contexts within knowledgebase 240, but where a new category and/or context results from the new concept 244, data processing engine 230 creates and adds an event 1206 to an appropriate input queue 1208 of event processing engine 1202. This is because update of categories and contexts within knowledgebase 240 are better handled when knowledgebase 240 is offline and not generating patient medical models 133, and other such outputs, since relationships between contexts 244 are recalculated to allow for the added concepts. Although shown with four input queues 1208, event processing engine 1202 may have more or fewer queues 1208 without departing from the scope hereof.

Periodically (e.g., when system 100 is less used, such as early morning or late at night), event processing engine 1202 takes knowledgebase 240 offline, processes events 1206 within event queues 1208 to create new categories and contexts within knowledgebase 240, and then puts knowledgebase 240 back online.

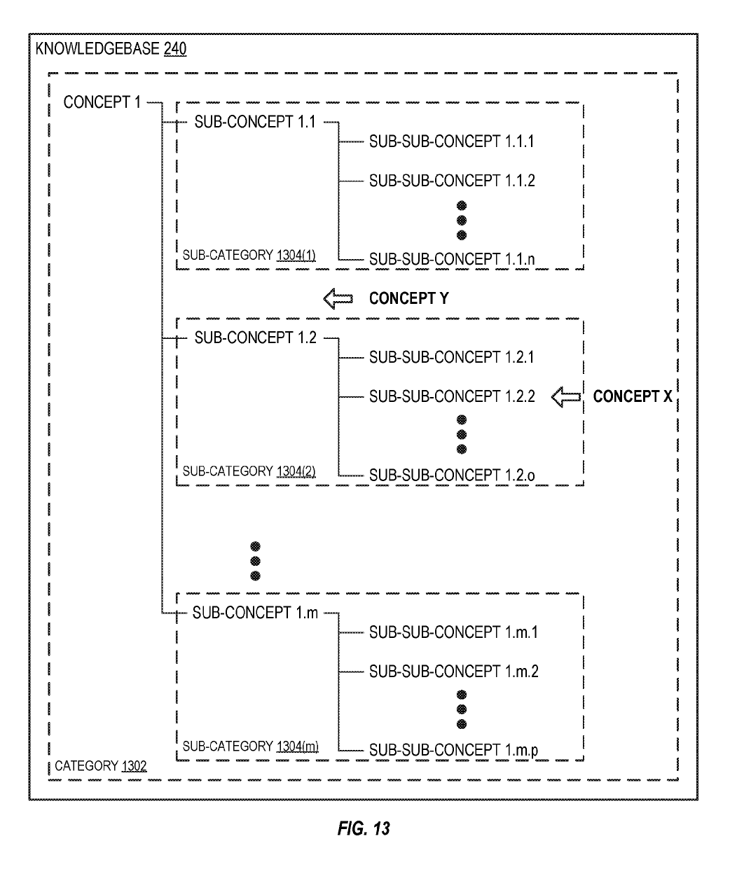

FIG. 13 shows knowledgebase 240 with exemplary data illustrating the ability to add concepts on the fly, and the need to create events 1206 to add concepts 244 within a new category. Knowledgebase 240 has a top level concept 1 within a category 1302. Concept 1 has a plurality of sub-concepts, sub-concept 1.1 through sub-concept 1.m. Sub-concept 1.1 is within a sub-category 1304(1), sub-concept 1.2 is within a sub-category 1304(2), and sub-concept 1.m is within a sub-category 1304(m). Sub-concept 1.1 has a plurality of sub-sub-concepts 1.1.1 through 1.1.n, each within a distinct sub-sub category of sub-category 1304(1). Sub-concept 1.2 has a plurality of sub-sub-concepts 1.2.1 through 1.2.o, each within a distinct sub-sub category of sub-category 1304(2). Sub-concept 1.m has a plurality of sub-sub-concepts 1.m.1 through 1.m.p, each within a distinct sub-sub-category of sub-category 1304(m).

Where a new concept X is determined by data processing engine 230, and it is determined to fall in the sub-sub-category corresponding to sub-sub-concept 1.2.2, concept X is added as another example of concepts within that sub-sub-category. Where a new concept Y is determined by data processing engine 230, and it is determined to fall within category 1302, and between sub-category 1304(1) and sub-category 1304(2) (i.e., not within an existing sub-category 1304), then data processing engine 230 generates and adds a new event 1206 to one queue 1208 of event processing engine 1202.

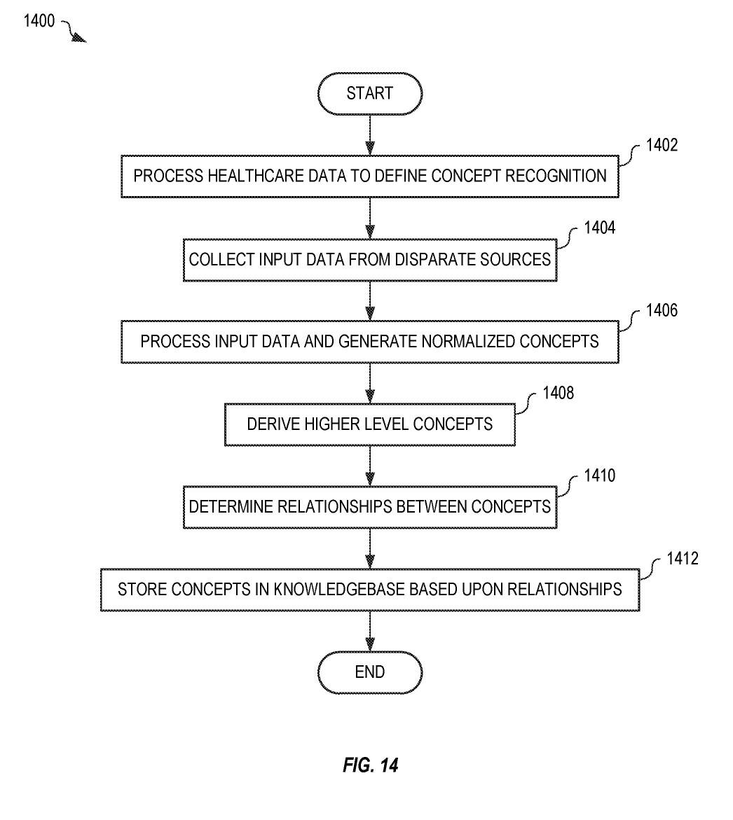

FIG. 14 is a flowchart illustrating one exemplary method 1400 for initializing system 100. Method 1400 is implemented within components of healthcare analytic engine 124. In step 1402, method 1400 processes healthcare data to define concept recognition. In one example of step 1402, phrase extraction and concept recognition tool 702 processes healthcare data 704 to “learn” phrase/word/keyword profiles 706, category profiles 708, concept profiles 710, and patient profiles 712, that specifically relate to the healthcare area. In step 1404, method 1400 collects input data from disparate sources. In one example of step 1404, healthcare analytic engine 124 receive, via transducers 131, healthcare information from disparate sources, such as a consulting room 104 of a doctor 105, a hospital 106, a laboratory 108, a pharmacy 110, a conventional electronic medical record database 160, the World Wide Web (WWW) 112.

In step 1406, method 1400 processes the input data and generates normalized concepts. In one example of step 1406, one or more data processing engines 230 process input data 120 to generate concepts 244. In step 1408, method 1400 derives higher level concepts. In one example of step 1408, analyzer 302 and/or data processing engine 230 generates concepts 612 from concepts 244 within knowledgebase 240. In step 1410, method 1400 determines relationships between concepts. In one example of step 1410, relationship engine 508 determines relationships between concepts 244 within knowledgebase 240. In step 1412, method 1400 stores concepts in the knowledgebase based upon the relationships. In one example of step 1412, relationship engine 508 stored concepts 244 within knowledgebase 240 based upon the determined relationships.

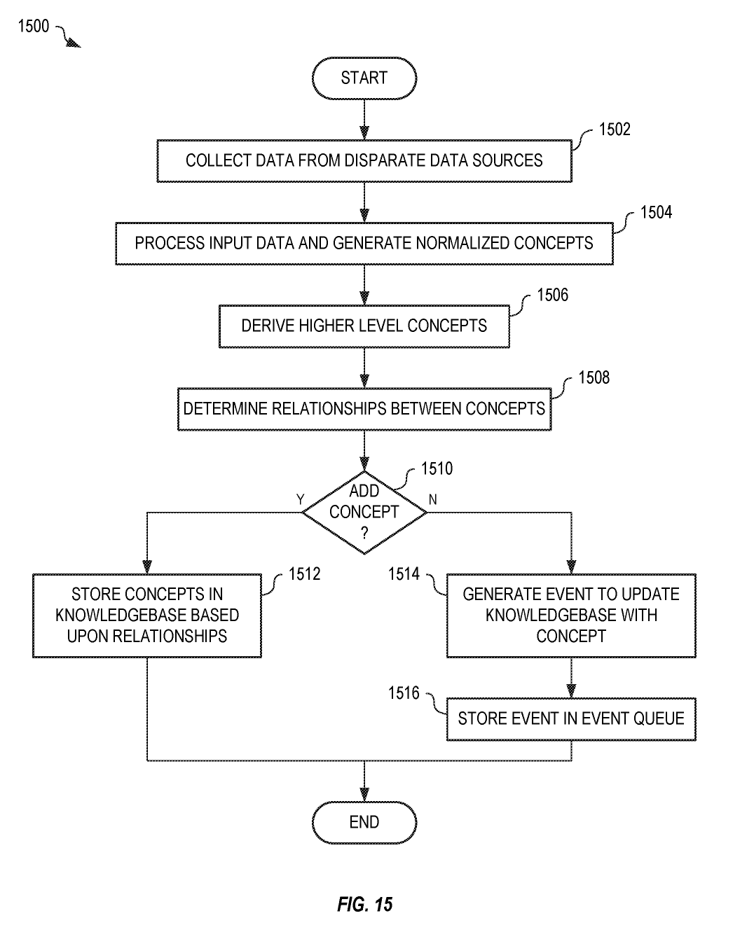

FIG. 15 is a flowchart illustrating one exemplary method 1500 for updating knowledgebase 240. Method 1500 is implemented within healthcare analytic engine 124 for example.

In step 1502, method 1100 collects data from disparate data sources. In one example of step 1502, healthcare analytic engine 124 receives input data 120 data from transducers 131 that collect healthcare information from disparate sources 103, 104, 106, 108, 110, 112, and 160. In step 1504, method 1500 processes the input data and generates normalized concepts. In one example of step 1504, data processing engine 230 processes input data 120 to determine one or more concepts 244. In step 1506, method 1500 derives higher level concepts. In one example of step 1506, analyzer 302 processes concepts 244 within knowledgebase 240 to generate high level concepts 612. In step 1508, method 1500 determines relationships between the concepts. In one example of step 1508, relationship engine 508 categorizes and determines relationships between concepts 244.

Step 1510 is a decision. If, in step 1510, method 1500 determines that the concept can be added to knowledgebase 240, method 1500 continues with step 1512; otherwise, method 1500 continues with step 1514. In step 1512, method 1500 stores the concept in the knowledgebase. In one example of step 1512, relationship engine 508 stores concepts 244 within knowledgebase 240 based upon the determined relationship of step 1508. Method 1500 then terminates. In step 1514, method 1500 generates an event to update the knowledgebase with the concept. In one example of step 1514, data processing engine 230 generates event 1206 based upon concept 244. In step 1516, method 1500 stores the event in the event queue. In one example of step 1516, data processing engine 230 adds event 1206 to queue 1208. Method 1500 then terminates.

Method 1500 repeats continually during normal operation of system 100.

### Medical Cost

Knowledgebase 240 may also store concepts 244 relating to cost of medical interventions performed on other patients, and may thereby predict cost of performing, and of not performing, similar medical interventions on patient 101. System 100 may thereby allow doctor 105 to select the most appropriate medical intervention for patient 101 and provide a cost estimate of not performing that intervention, or of postponing that intervention. For example, an omitted or postponed intervention may result in much larger medical costs at a later time for patient 101. Cost may also be based upon the insurance provider of patient 101. System 100 may thereby show doctor 105 and patient 101 how costs may be reduced and money saved.

### Example Implementation

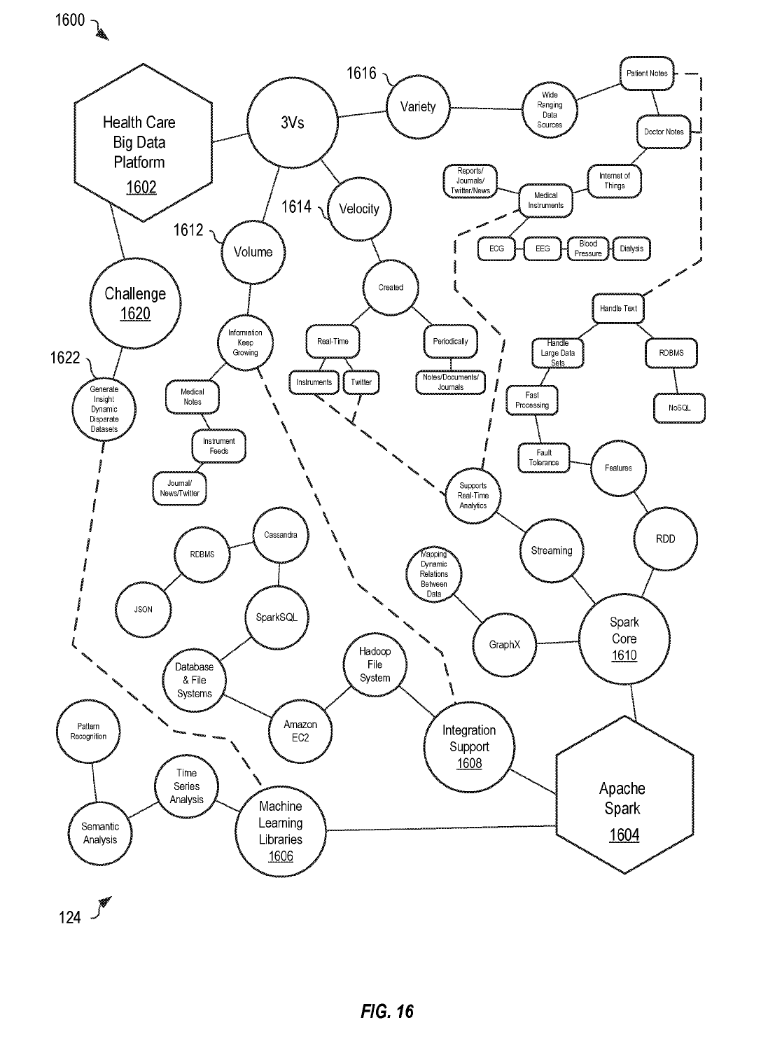

FIG. 16 shows one exemplary framework 1600 for implementing healthcare analytic engine 124 of FIGS. 1 and 2 using an Apache Spark platform, in an embodiment. Framework 1600 depicts health care big data's 3Vs and expands them with health care examples.

A healthcare big-data platform 1602 is shown at the top left of FIG. 1 and a ‘generic’ Apache Spark 1604 is shown at the bottom right. Framework 1600 includes three main hubs: machine learning libraries 1606, integration support 1608 and Spark core 1610. These hubs translate each of the three goals of a big-data platform: volume 1612, velocity 1614, and variety 1616.

Volume 1612 represents a huge volume of data received in various forms such as medical notes, and instrument feeds, to name a few, often received in time series or as continuous feed, and other data sources. This received data is stored, normalized, harvested and eventually ingested using framework 1600. These requirements are translated using Integration Support 1608. In this example embodiment, database 202 is primarily implemented using Cassandra and uses the Hadoop File System hosted on an Amazon EC2 Virtual instance. Cassandra allowing queries to be run using SparkSQL and also provides support with standard data transport protocols such as JSON as may be used to transport data in FIG. 1 of Appendix B of U.S. Patent Application Ser. No. 62/194,920.

### Velocity

Healthcare big-data platform 1602 supports real time data, which may be periodic or asynchronous, and functionality for processing these types of data is realized by exploiting the real time processing framework of Apache Spark 1604. For example, real-time feeds from various medical instruments, such as ECG, EEG, Blood Pressure Monitors or Dialysis Machines, shown as transducers 231 of system 100 in FIG. 2 of Appendix A of U.S. Patent Application Ser. No. 62/194,920.

### Variety

Healthcare big-data platform 1602 supports data from disparate sources that is handled by our big data platform. These are processed by translating them through various modules that connects with ‘core’ Spark modules. One such example is patient notes that contain natural language phrases 602 as shown in FIG. 6 of Appendix A of U.S. Patent Application Ser. No. 62/194,920. These modules include text handler, query processor (e.g., see FIG. 7 of Appendix A of US Patent Application Ser. No. 62/194,920) and NoSQL database support. Another example is Speech Processing and Analysis as shown in FIG. 5. These are mapped using a Resilient Distributed Data Set framework as supported by Apache Spark 1604.

### Big Data Analytics

Machine Learning Library 1606 provides access to standard machine learning algorithms such as pattern recognition, time series analysis, and semantic analysis. These algorithms may be used to process data from transducers 231 of FIGS. 2 and 3 of Appendix A of U.S. Patent Application Ser. No. 62/194,920, big data 450 of FIG. 4, and phrase extraction and concept recognition tool 702 of FIG. 7 for example. Framework 1600 thereby implements intelligence of analytic engine 224 of FIGS. 2, 4 and 5 of Appendix A of U.S. Patent Application Ser. No. 62/194,920, healthcare analytic engine 124 of FIGS. 1, 2, and 3, and analytic engine 124 of FIG. 1 of Appendix B of U.S. Patent Application Ser. No. 62/194,920. This described functionality is implemented by framework 1600 to overcome one of the biggest challenges 1620, how to process and generate insight from multiple disparate data sources 1622 within Healthcare big data platform 1602.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween. In particular, the following embodiments are specifically contemplated, as well as any combinations of such embodiments that are compatible with one another:

(A1) A method for analyzing healthcare data, including collecting first input data from a first source; collecting second input data from a second source disparate from the first source, the second source having a data format that is different from a format of the first source; processing the first input data to determine a first concept; processing the second input data to determine a second concept; determining a relationship between the first and second concepts; storing the first and second concepts within a knowledgebase based upon the relationship; and generating a patient medical model from the knowledgebase.

(A2) In the method for analyzing healthcare data denoted above as (A1), the step of processing the first input data comprising normalizing healthcare data within the first input data based upon a healthcare matrix; and the step of processing the second input data comprising normalizing the healthcare data within the second input data based upon the healthcare matrix; wherein the first and second concepts have a format that allows comparison.

(A3) In either method for analyzing healthcare data denoted above as (A1)-(A2), the step of determining the relationship comprising determining a healthcare category for each of the first and second concepts, the relationship being based upon the healthcare categories.

(A4) In any of the methods for analyzing healthcare data denoted above as (A1)-(A3), wherein at least one of the first input data and the second input data comprises non-verbal information.

(B1) A method for analyzing healthcare data, including: receiving input data from a plurality of disparate sources; extracting text from the input data; processing the text using natural language processing (NLP) to determine a plurality of concepts, each concept based upon understanding and sentiment derived from the text; determining a relationship between each of the concepts; deriving high level concepts from the plurality of concepts; storing each of the concepts and the high level concepts within a database based upon the relationship; processing the input data to determine concepts relating to healthcare; normalizing the information in each of the concepts; extracting direct concepts from the healthcare data by using NLP, semantic analysis, and inference extraction; deriving derived concepts from the direct concepts; and storing the direct concepts and the derived concepts in a concept bank to form a knowledgebase.

(B2) In either method for analyzing healthcare data denoted above as (B1), the step of determining the relationship including determining context for each of the concepts; and determining a category for each of the concepts; wherein the relationship is based upon one or both of the context and the category.

(B3) In either method for analyzing healthcare data denoted above as (B1)-(B2), further comprising processing the knowledgebase to forecast patient behaviors and healthcare events.

(B4) In any of the methods for analyzing healthcare data denoted above as (B1)-(B3), the step of processing including: selecting certain concepts from the concept bank; plotting the concepts on a concept graph; and processing the concept graph to forecast the patient behaviors and healthcare events.

(B5) In any of the methods for analyzing healthcare data denoted above as (B1)-(B4), further including periodically repeating the steps of receiving, extracting, deriving, and storing to maintain the concept bank.

(B6) In any of the methods for analyzing healthcare data denoted above as (B1)-(B5), further including retrieving healthcare data from a plurality of internet sources, the databases including healthcare data learning.

(B7) In any of the methods for analyzing healthcare data denoted above as (B1)-(B6), the step of normalizing including normalizing the information within the concept based upon a healthcare matrix.

(B8) In any of the methods for analyzing healthcare data denoted above as (B1)-(B7), the input data including both verbal and non-verbal information.

(B9) In any of the methods for analyzing healthcare data denoted above as (B1)-(B8), the data being at least one of asked data, evoked data, detected data, symptom data, sign data, lab data, imaging data, test data, and sensory data.

(C1) A system for analyzing healthcare data including a plurality of transducers operable to collect healthcare data from disparate sources; a natural language processing (NLP) and semantic engine for identifying direct concepts in the healthcare data; a converter, implemented as machine readable instruction executed by a digital processor, for receiving and converting the healthcare data to form a database of information associated with the patient; and an analyzer, implemented as machine readable instruction executed by a digital processor, for processing the database to generate a health status of the patient.

(C2) In the system for analyzing healthcare data denoted above as (C1), further including a trigger rules engine for identifying the direct concepts based upon language rules specific to the language of the healthcare data.

(D1) A software product comprising instructions, stored on non-transitory computer-readable media, wherein the instructions, when executed by a computer, perform steps for analyzing healthcare data, including: instructions for collecting first input data from a first source; instructions for collecting second input data from a second source disparate from the first source, the second source having a data format that is different from a format of the first source; instructions for processing the first input data and the second input data to determine a first concept and a second concept, respectively; instructions for determining a relationship between the first and second concepts; instructions for storing the first and second concepts within a knowledgebase based upon the relationship; and instructions for generating a patient medical model from the knowledgebase.

(D2) In the software product denoted above as (D1), the instructions for processing the first input data comprising instructions for normalizing healthcare data within the first input data based upon a healthcare matrix; and the instructions for processing the second input data comprising instructions for normalizing the healthcare data within the second input data based upon the healthcare matrix; wherein the first and second concepts have a format that allows comparison.

(D3) In either of the software products denoted above as (D1) and (D2), the instructions for determining the relationship comprising instructions for determining a healthcare category for each of the first and second concepts.

(D4) In any of the software products denoted above as (D1)-(D3), wherein at least one of the first input data and the second input data comprises non-verbal information.

(D5) In any of the software products denoted above as (D1)-(D4), at least one of the first input data and second input data comprising at least one of asked data, evoked data, detected data, symptom data, sign data, lab data, imaging data, test data, as well as sensory data.

(E1) A software product comprising instructions, stored on non-transitory computer-readable media, wherein the instructions, when executed by a computer, perform steps for analyzing healthcare data, including: instructions for receiving input data from a plurality of disparate sources; instructions for extracting text from the input data; instructions for processing the text using natural language processing (NLP) to determine a plurality of concepts, each concept based upon understanding and sentiment derived from the text; instructions for determining a relationship between each of the concepts; instructions for storing each of the concepts and the high level concepts within a database based upon the category; instructions for deriving high level concepts from the plurality of concepts; instructions for storing each of the concepts and the high level concepts within a database based upon the category; instructions for processing the input data to determine concepts relating to healthcare; instructions for normalizing the information in each of the concepts; instructions for extracting direct concepts from the healthcare data by using NLP, semantic analysis, and inference extraction; instructions for deriving derived concepts from the direct concepts; and instructions for storing the direct concepts and the derived concepts in a concept bank to form a knowledgebase.

(E2) In the software product denoted above as (E1), the instructions for determining the relationship including: instructions for determining context for each of the concepts; and instructions for determining a category for each of the concepts; wherein the relationship is based upon one or both of the context and the category.

(E3) In either software product denoted above as (E1) and (E2), further including instructions for periodically repeating the steps of receiving, extracting, deriving, and storing to maintain the concept bank.

(E4) In any of the software products denoted above as (E1)-(E3), further including instructions for retrieving healthcare data from a plurality of internet sources, the databases including healthcare data learning.

(E5) In any of the software products denoted above as (E1)-(E4), the instructions for normalizing including instructions for normalizing the information within the concept based upon a healthcare matrix.

(E6) In any of the software products denoted above as (E1)-(E5), wherein the input data comprises both verbal and non-verbal information.

(E7) In any of the software products denoted above as (E1)-(E6), the input data being at least one of asked data, evoked data, detected data, symptom data, sign data, lab data, imaging data, test data, as well as sensory data.

What is claimed is:

1. A method for analyzing healthcare data, comprising:
collecting first input data from a first source;
collecting second input data from a second source disparate from the first source, the second source having a data format that is different from a format of the first source;
processing the first input data to determine a first concept;
processing the second input data to determine a second concept;
determining a relationship between the first and second concepts;
storing the first and second concepts within a knowledgebase based upon the relationship; and
generating a patient medical model from the knowledgebase.

2. The method of claim 1, the step of processing the first input data comprising normalizing healthcare data within the first input data based upon a healthcare matrix; and the step of processing the second input data comprising normalizing the healthcare data within the second input data based upon the healthcare matrix; wherein the first and second concepts have a format that allows comparison.

3. The method of claim 1, the step of determining the relationship comprising determining a healthcare category for each of the first and second concepts, the relationship being based upon the healthcare categories.

4. The method of claim 1, wherein at least one of the first input data and the second input data comprises non-verbal information.

5. A method for analyzing healthcare data, comprising:
receiving input data from a plurality of disparate sources;
extracting text from the input data;
processing the text using natural language processing (NLP) to determine a plurality of concepts, each concept based upon understanding and sentiment derived from the text;
determining a relationship between each of the concepts;
deriving high level concepts from the plurality of concepts;
storing each of the concepts and the high level concepts within a database based upon the relationship;
processing the input data to determine concepts relating to healthcare;
normalizing the information in each of the concepts;
extracting direct concepts from the healthcare data by using NLP, semantic analysis, and inference extraction;
deriving derived concepts from the direct concepts; and
storing the direct concepts and the derived concepts in a concept bank to form a knowledgebase.

6. The method of claim 5, the step of determining the relationship comprising:
determining context for each of the concepts; and
determining a category for each of the concepts;
wherein the relationship is based upon one or both of the context and the category.

7. The method of claim 5, further comprising processing the knowledgebase to forecast patient behaviors and healthcare events.

8. The method of claim 7, the step of processing comprising:
selecting certain concepts from the concept bank;
plotting the concepts on a concept graph; and
processing the concept graph to forecast the patient behaviors and healthcare events.

9. The method of claim 5, further comprising periodically repeating the steps of receiving, extracting, deriving, and storing to maintain the concept bank.

10. The method of claim 5, further comprising retrieving healthcare data from a plurality of internet sources, the databases comprising healthcare data learning.

11. The method of claim 5, the step of normalizing comprising normalizing the information within the concept based upon a healthcare matrix.

12. The method of claim 5, wherein the input data comprises both verbal and non-verbal information.

13. The method of claim [[4]] 5, the data being at least one of asked data, evoked data, detected data, symptom data, sign data, lab data, imaging data, test data, and sensory data.

14. A system for analyzing healthcare data, comprising:
a plurality of transducers operable to collect healthcare data from disparate sources;
a natural language processing (NLP) and semantic engine for identifying direct concepts in the healthcare data;
a converter, implemented as machine readable instruction executed by a digital processor, for receiving and converting the healthcare data to form a database of information associated with the patient; and
an analyzer, implemented as machine readable instruction executed by a digital processor, for processing the database to generate a health status of the patient.

15. The system of claim 14, further comprising a trigger rules engine for identifying the direct concepts based upon language rules specific to the language of the healthcare data.

16-29. (canceled)

30. The system of claim 14, the analyzer further comprising machine readable instruction executed by the digital processor, for:
deriving derived concepts from the direct concepts; and
storing the direct concepts and the derived concepts in a concept bank to form a knowledgebase.

31. The system of claim 30, the analyzer further comprising machine readable instruction executed by the digital processor, for processing the knowledgebase to forecast patient behaviors and healthcare events.

32. The system of claim 30, the analyzer further comprising machine readable instruction executed by the digital processor, for:
selecting certain concepts from the concept bank;
plotting the concepts on a concept graph; and
processing the concept graph to forecast the patient behaviors and healthcare events.

33. The system of claim 30, the converter further comprising machine readable instruction executed by the digital processor, for:
determining context for each of the concepts;
determining a category for each of the concepts; and
determining a relationship between concepts based upon one or both of the context and the category.

34. The system of claim 30, the healthcare data comprising at least one of asked data, evoked data, detected data, symptom data, sign data, lab data, imaging data, test data, as well as sensory data.
